(19) United States
(12) Patent Application Publication
(10) Pub. No.: US 2025/0205393 A1
(43) Pub. Date: Jun. 26, 2025

(54) COMPOSITIONS FOR MINERALIZED TISSUES REPAIR AND REGENERATION

(71) Applicants: SORBONNE UNIVERSITE, PARIS (FR); CENTRE NATIONAL DE LA RECHERCHE SCIENTIFIQUE (CNRS), PARIS (FR)

(72) Inventors: Nadine NASSIF, PARIS CEDEX 05 (FR); Miléna LAMA, PARIS CEDEX 05 (FR); Camila BUSSOLA TOVANI, PARIS CEDEX 05 (FR); Marc ROBIN, PARIS CEDEX 05 (FR)

(73) Assignees: SORBONNE UNIVERSITE, PARIS (FR); CENTRE NATIONAL DE LA RECHERCHE SCIENTIFIQUE (CNRS), PARIS (FR)

(21) Appl. No.: 18/849,374

(22) PCT Filed: Mar. 23, 2022
(86) PCT No.: PCT/IB2022/000162

§ 371 Date: Sep. 20, 2024

(51) Int. Cl. A61L 27/46; A61L 27/24

(52) U.S. Cl. CPC A61L 27/46; A61L 27/24; A61L 2400/06; A61L 2430/02

(57) ABSTRACT

The present disclosure relates to injectable or implantable compositions that are able to mimic different types of mineralized biological tissues and their uses in mineralized tissues repair and regeneration.

### FIELD OF THE INVENTION

The present invention relates to the field of repair and regeneration of mineralized biological tissues. In particular, it relates to injectable or implantable compositions that are able to mimic different types of mineralized biological tissues and their uses in mineralized tissues (e.g., bones) repair and regeneration.

### BACKGROUND OF THE INVENTION

Bone is a hybrid tissue. The extracellular matrix (ECM) of bone is composed of a mineral phase (about 65 wt %), an organic phase (around 25 wt %) with type I collagen as main component, and water (about 10 wt %). This ECM is continuously renewed and remodeled allowing the repair of small defects (such as micro fractures) and tissue mechanical adaptation. However, when the defect is too large, it can no longer be resorbed by cellular processes (Schmitz, J. P. and J. O. Hollinger. 1986. “The Critical Size Defect as an Experimental Model for Craniomandibulofacial Nonunions.” Clinical Orthopaedics and Related Research NO. 205:299-308). A scaffold (support) is then needed to promote bone repair. Ideally, the scaffold has the same properties as bone tissues (composition, ultrastructure, mechanical properties).

Bone autografts remain the gold standard for bone grafting (Bauer, Thomas W. and George F. Muschler. 2000. “Bone Graft Materials.” Clinical Orthopaedics and Related Research 371:10-27). However, bone autograft materials have several drawbacks such as limited availability and the need to perform an additional surgery that could worsen the patient's condition. In order to limit the invasiveness of two-steps surgical procedures, injectable hybrid materials for bone regeneration have been proposed such as hydroxyapatite microparticles coated with low concentrated collagen (Flautre, B. et al. (1996) ‘Evaluation of hydroxyapatite powder coated with collagen as an injectable bone substitute: Microscopic study in rabbit’, Journal of Materials Science: Materials in Medicine, 7(2), pp. 63-67) or mineralized low concentrated collagen microspheres (Yin Hsu, F., Chueh, S. C. and Jiin Wang, Y. (1999) ‘Microspheres of hydroxyapatite/reconstituted collagen as supports for osteoblast cell growth’, Biomaterials, 20(20), pp. 1931-1936). Yet, these methods systematically require a chemical crosslinking step due to the fragility of the microspheres owing to their low collagen concentration, which may lead to inflammation and poor biological response e.g., osseointegration (Speer, D. P., Chvapil, M., Eskelson, C. & Ulreich, J. Biological effects of residual glutaraldehyde in glutaraldehyde-tanned collagen biomaterials. J. Biomed. Mater. Res. 14, 753-764 (1980); Bellincampi, L. D. & Dunn, M. G. Effect of crosslinking method on collagen fiber-fibroblast interactions. J. Appl. Polym. Sci. 63, 1493-1498 (1997)). Another strategy consists in mineralizing collagen fibrils to form an injectable paste (Liu, X. et al. (2010) ‘Injectable bone cement based on mineralized collagen’, Journal of Biomedical Materials Research—Part B Applied Biomaterials, 94(1), pp. 72-79; Pelin, I. M. et al. (2009) ‘Preparation and characterization of a hydroxyapatite-collagen composite as component for injectable bone substitute’, Materials Science and Engineering C. Elsevier B. V., 29(7), pp. 2188-2194). Here again, low concentrated collagen is used which prevents the formation of a bone-like 3D architecture because the threshold for collagen self-assembly is not reached (Giraud-Guille, Marie Madeleine. 1992. “Liquid Crystallinity in Condensed Type I Collagen Solutions. A Clue to the Packing of Collagen in Extracellular Matrices.” Journal of Molecular Biology 224(3): 861-73; Giraud-Guille, Marie Madeleine and Laurence Besseau. 1998. “Banded Patterns in Liquid Crystalline Phases of Type I Collagen: Relationship with Crimp Morphology in Connective Tissue Architecture.” Connective Tissue Research 37(3-4):183-93). In addition, none of the cited works use biomimetic hydroxyapatite in terms of composition, structure, morphology, size (Nassif, N., F. Martineau, O. Syzgantseva, F. Gobeaux, M. Willinger, T. Coradin, S. Cassaignon, T. Azaïs, and M. M. Giraud-Guille. 2010. “In Vivo Inspired Conditions to Synthesize Biomimetic Hydroxyapatite.” Chemistry of Materials 22(12):3653-63) and properties when hydrated (Wang, Yan, Stanislas Von Euw, Francisco M. Fernandes, Sophie Cassaignon, Mohamed Selmane, Guillaume Laurent, Gérard Pehau-Arnaudet, Cristina Coelho, Laure Bonhomme-Coury, Marie Madeleine Giraud-Guille, Florence Babonneau, Thierry Azaïs, and Nadine Nassif. 2013. “Water-Mediated Structuring of Bone Apatite.” Nature Materials 12(12): 1144-53). In order to improve the mechanical properties of such biomaterials for an easier application in the defect i.e. adhesion and maneuverability, other formulations with additives have been proposed (Chen, Z. et al. (2011) ‘Injectable calcium sulfate/mineralized collagen-based bone repair materials with regulable self-setting properties’, Journal of Biomedical Materials Research—Part A, 99 A(4), pp. 554-563; Huang, Z. et al. (2009) ‘A bone-like nano-hydroxyapatite/collagen loaded injectable scaffold’, Biomedical Materials, 4(5)). They enable a good injectability of the biomaterial and an in situ gelation enabling to fill the defect within a short time span. Nevertheless, the composition of such materials is not biomimetic, thus preventing the reproduction of bone ultrastructure.

In fact, the 3D organization of highly concentrated collagen materials appears as a sine qua non condition to mimic the ultrastructure of biological tissues, in particular the twisted plywood found in bone (Wang, Y. et al. (2011) ‘Controlled collagen assembly to build dense tissue-like materials for tissue engineering’, Soft Matter, 7(20), pp. 9659-9664), and to promote colonization by host cells. Such fibrillar collagen matrices also exhibit improved mechanical properties (Lama, M, et al. “Biomimetic Tough Gels with Weak Bonds Unravel the Role of Collagen from Fibril to Suprafibrillar Self-Assembly.” Macromolecular Bioscience 21.6 (2021): 2000435) without the need of additives or crosslinking agents. Previous works showed that the mineralization of highly concentrated collagen matrices promotes both the formation of biomimetic hydroxyapatite nanoplatelets and their co-alignment with collagen fibrils (Wang, Y. et al. (2012) ‘The predominant role of collagen in the nucleation, growth, structure and orientation of bone apatite.’, Nature materials. Nature Publishing Group, 11 (8), pp. 724-33.). Even if such matrices are less mineralized than native bone tissue, this degree of order was shown to lead to stiffnesses reaching gigapascals (Nassif, N. et al. (2010) ‘In vivo inspired conditions to synthesize biomimetic hydroxyapatite’, Chemistry of Materials, 22(12), pp. 3653-3663), thus strengthening the interest in processing high collagen concentrations. However, such pre-formed collagen matrices are not injectable.

Therefore, it remains a need for an injectable or implantable composition that overcomes one or more of the above-mentioned drawbacks, in particular it remains a need for an injectable or implantable composition that is able to mimic different types of mineralized biological tissues, in particular bone, and that exhibits good cohesion and adhesion to the defect to be repaired, allowing rapid mineralized tissue regeneration.

### SUMMARY OF THE INVENTION

The invention relates to a composition comprising:
collagen microparticles comprising more than 90% by weight of collagen;
biomimetic hydroxyapatite or biomimetic hydroxyapatite precursors or amorphous calcium phosphate; and
a physiologically compatible aqueous solvent.

The invention also relates to a composition as defined herein for use in mineralized tissue repair and regeneration, for inducing new bone formation, promoting bone growth and/or treating bone defects and for use in repairing bones defects in bone reconstructive procedure, preferably in maxillofacial surgery or orthopaedic surgery.

The invention also relates to a preformed implantable matrix comprising a composition as described herein.

The invention also relates to processes as recited in claims 13, 14 and 15 for preparing a composition as described herein.

Further aspects of the invention are as disclosed herein and in the claims.

### FIGURES

FIG. 1: TGA thermogram of a hybrid collagen material showing good agreement between initial weights (collagen microparticles contain about 10 wt % water) and measured organic and inorganic contents (initial collagen/hydroxyapatite ratio 1:1).

FIG. 2: DSC analysis of different collagen materials prepared with saline solution displaying similar endothermal peaks typical of collagen denaturation.

FIG. 3: PLM observations of a hybrid collagen solution: bright birefringent textures evidence anisotropic organizations.

FIG. 4: SEM micrograph of a mineralized collagen material displaying partially dissolved collagen microparticles, before fibrillogenesis (left). After fibrillogenesis (right) the material exhibits more defined collagen fibrils.

FIG. 5: SEM micrograph of a mineralized collagen gel (collagen/hydroxyapatite ratio 1:1) displaying fibrillar alignment domains in a dense matrix.

FIG. 6: TEM micrograph of unstained ultrathin section of a collagen/HA 50:50 matrix with high dry matter content displaying co-alignment of collagen fibrils and hydroxyapatite nanoplatelets.

FIG. 7: Image illustrating the presence of fibrovascular and leukocytic infiltration/colonisation within a fragment of injectable material. The circles identify the presence of infiltrates/cell colonies within a fragment of injectable material (*). (□) polymorphic inflammatory reaction with macrophagic and multinucleated giant cell component in contact with the material.

FIG. 8: Pre-formed material (process 2) produced using dense collagen microparticles mixed with biomimetic hydroxyapatite precursors solution. (a-c) SEM micrograph showing that the material displays a high density of collagen fibrils. (d) TEM micrograph showing the alignment of collagen fibrils in the material. (e) Infrared spectra of (i) dense collagen microparticles, (ii) pre-formable material (process 2) produced using dense collagen microparticles mixed with biomimetic hydroxyapatite precursors solution and (iii) biomimetic apatite (CHA). It can be observed that the preformed material displays vibrational bands ascribed to both collagen and apatite, thus confirming the formation of a collagen mineralized matrix.

FIG. 9: Hybrid dense collagen/biomimetic hydroxyapatite precursors microparticles (process 3). (a) SEM micrograph of hybrid dense microparticles. (b) EDX spectra showing the incorporation of biomimetic hydroxyapatite precursors in the dense collagen microparticles. (c-d) SEM micrograph of pre-formed material produced using the hybrid dense collagen/biomimetic hydroxyapatite precursors microparticles. A high density of fibrils can be observed.

### DESCRIPTION OF THE INVENTION

The inventors have developed compositions that overcome one or more of the above-mentioned drawbacks. They allow the injection or implantation of mineralized, highly concentrated collagen matrix with bone-like features in terms of composition and ultrastructure. The compositions developed by the inventors are able to mimic different types of mineralized biological tissues. They preserve collagen self-assembly properties and even promote collagen and hydroxyapatite biomimetic co-assembly. The compositions are biocompatible and favor host cell colonization. They do not trigger inflammatory response. Such compositions pave the way for biomimetic and injectable mineralized tissues substitutes, such as bone, with adaptable compositions. They may be used in the field of bone tissue regeneration and offer a promising new therapeutic way for efficient tissue regeneration.

The compositions developed by the inventors comprise:
dense collagen microparticles (i.e. microparticles comprising more than 90 wt % of collagen);
biomimetic hydroxyapatite or biomimetic hydroxyapatite precursors or amorphous calcium phosphate; and
a physiologically compatible aqueous solvent.

The compositions are suitable for injection and/or implantation. The compositions may then be defined as being injectable and/or implantable.

The components of the compositions of the present invention are as described in details herein below.

The term <<injectable>as used herein designates the ability to place the material in the site of interest by means of a commercial syringe with or without a needle.

The term « implantable >as used herein designates an object capable of being implanted in a person's body by conventional surgical procedures.

### Dense Collagen Microparticles

The term “dense collagen microparticles” as used herein designates collagen microparticles comprising more than 90% by weight of collagen, in particular more than 91%, 92%, 93%, 94%, 95%, 96%, 97%, 98% by weight of collagen, the remaining being water.

The dense collagen microparticles are as disclosed in WO2016/146954.

The dense collagen microparticles are in the form of solid spherical or spheroid particles formed of non-denatured and uncrosslinked collagen. The diameter of the particles typically ranges from 0.05 to 20 μm, in particular from 0.25 to 10 μm, more particularly from 0.4 μm to 3 μm. It is to be understood that the particles diameter ranges refer to the diameter distribution. The particles typically have a diameter ranging from a minimum diameter of 0.05 μm to a maximum diameter of 20 μm.

The term “spheroid” as used herein designates a solid of which the shape assimilates to that of a sphere.

The term “diameter” designates the diameter of the sphere or the greatest diameter of the spheroid. The diameter can be measured for example by electron microscopy or by dynamic light scattering.

The term “non-denatured” as used herein designate a collagen of which the secondary structure of the α-triple helices is preserved. The non-denatured or denatured nature of collagen can be observed for example by calorimetric analysis. Denatured collagen has a calorimetric profile characteristic of a denatured protein (gelatin), with no sign of organized macromolecular domains.

The term “uncrosslinked” as used herein designates a collagen in which there are no crosslinking bonds, whether these bonds are the result of chemical, such as treatment by glutaraldehyde, or enzymatical or physical modifications. The absence of crosslinking can be determined for example by electrophoresis.

The dense collagen microparticles may be prepared from a variety of collagen. Hence, the source of collagen is irrelevant. The collagen can be obtained in accordance with the following protocol: a solution of type I collagen is prepared from Wistar rat tail tendons. After excision in a laminar flow cabinet, the tendons are washed in a sterile saline phosphate buffer solution. The tendons are then immersed in a solution of 4 M NaCl in order to remove the remaining intact cells and precipitate some of the proteins of elevated molecular weight. After washing by the saline phosphate buffer solution, the tendons are solubilized in a sterile 500 mM acetic acid solution. The solution obtained is clarified by centrifugation at 41000 g for 2 hours. The proteins other than the collagen are precipitated selectively in an aqueous solution of 300 mM NaCl and removed by centrifugation at 41000 g for 3 hours. The collagen is recovered from the supernatant by precipitation in a solution of 600 mM NaCl followed by centrifugation at 3000 g for 45 minutes. The pellets obtained are solubilized in an aqueous solution of 500 mM acetic acid, then dialysed in the same solvent in order to remove the NaCl ions. The solution is held at 4° C. and centrifuged at 41000 g for 4 hours prior to use. This detailed protocol can be applied to other types of collagen.

The collagen of the dense collagen microparticles has typically a molecular mass ranging from 200 to 450 KDa.

The collagen of the dense collagen microparticles is typically a type I collagen. Nevertheless, the collagen may alternatively be of type II, III, V, XI, XXIV, XXVII, and mixtures thereof.

The dense collagen microparticles may be prepared by a spray-processing technology as disclosed in WO2016/146954. In brief, the spray-processing technology consists in atomizing an acid-soluble collagen solution (non-denatured and uncrosslinked collagen) in order to form a mist of very thin droplets, immediately dried by evaporation of the solvent in a controlled atmosphere (thanks to the high solution/air interface area of the droplets). The concentration of collagen in the acidic collagen solution typically ranges from 0.1 to 10 mg/L. The acidic collagen solution has a pH inferior to 7. The acid is typically acetic acid. The acetic acid concentration in the acidic collagen solution typically ranges from 0.1 to 1000 mM. The atomization is typically performed at a temperature below about 40° C., in particular below about 39° C., 38° C. or 37° C., to obtain a powdered composition. The concentration in the collagen drops is high enough to induce the self-assembly of collagen molecules and a subsequent liquid crystal order, e.g. nematic oriented domains. This strategy allows obtaining within seconds highly concentrated collagen microparticles circumventing the high increase of viscosity of type I collagen solutions that usually prevents fast processing of this protein, and consequently its use at biological concentration.

It was previously shown that the formation of dense collagen microparticles by aerosol (WO2016/146954) allowed the injection of highly concentrated collagen gels (at least up to 80 mg/mL) (Milena Lama, Francisco M. Fernandes, Alba Marcellan, Juliette Peltzer, Marina Trouillas, Sébastien Banzet, Marion Grosbot, Clément Sanchez, Marie Madeleine Giraud-Guille, Jean Jacques Lataillade, Bernard Coulomb, Cédric Boissière, and Nadine Nassif. 2020. “Self-Assembled Collagen Microparticles by Aerosol as a Versatile Platform for Injectable Anisotropic Materials.” Small 16 (4): 1-8). After injection or setting in a mold (Lama, M, et al. “Biomimetic Tough Gels with Weak Bonds Unravel the Role of Collagen from Fibril to Suprafibrillar Self-Assembly.” Macromolecular Bioscience 21.6 (2021): 2000435), the self-assembled collagen matrix exhibited tissue-like features both in terms of ultrastructure and mechanical properties.

### Biomimetic Hydroxyapatite

The terms “biomimetic hydroxyapatite” refer to bone-like hydroxyapatite platelets.

The biomimetic hydroxyapatite is typically in the form of powder.

The biomimetic hydroxyapatite powder may be synthesized following a procedure described by Nassif et al., Chemistry of Materials, 22 (12), pp.3653-3663, 2010. Briefly, biomimetic hydroxyapatite is prepared via vapor diffusion of ammonia (NH₃) into an acidic calcium-phosphate (CaCl₂—NaH₂PO₄— or possibly with other salts in particular NaHCO₃) solution based on thermodynamic conditions to avoid the precipitation of other calcium-phosphate phases. For instance, biomimetic hydroxyapatite may be prepared by precipitation of a CaCl₂/NaH₂PO₄ acidic solution (acetic acid, 500 mM) with a calcium-to-phosphate (Ca/P) molar ratio which is consistent with the formation of hydroxyapatite with a formula of Ca₁₀(PO₄)₆(OH₂) or of a CaCl₂/NaH₂PO₄/NaHCO₃ acidic solution (acetic acid, 500 mM) with a calcium-to-phosphate plus carbonate (Ca/[P+C]) molar ratio which is consistent with the formation of hydroxyapatite with a formula of Ca₁₀₋ₓ(PO₄)₆₋ₓ(CO₃)ₓ(OH)₂₋ₓ with 0≤x≤2. The precipitation is triggered by the addition of an ammonia aqueous solution (30%, w/w).

This precipitation method, which is free of any organic additives, has the advantage of being conducted at room temperature within a few hours, without direct pH control, and does not produce any by-product or non-desired (i.e. non-physiological) phases. It was shown that the synthesis of biomimetic hydroxyapatite as disclosed by Nassif et al., 2010 results in nanoplatelets exhibiting similar self-assembling properties in water as native bone apatites (Wang, Yan, et al. “Water-mediated structuring of bone apatite.” Nature materials 12.12 (2013): 1144-1153). The nanoplatelets have been shown to have a crystalline core and amorphous shell with X-ray diffraction pattern matching that of JCPDS N 9-0432. They typically have an average size of 200×100×5 nm³ and carbonate substitution as observed for bone mineral.

It should be noted that the composition of hydroxyapatite can also be modified and in particular enriched with strontium (up to 10% Calcium substitution) to combine anti-osteoporotic effects (Tovani et al. ‘Formation of stable strontium-rich amorphous calcium phosphate: Possible effects on bone mineral’, Acta biomaterialia, 2019).

### Biomimetic Hydroxyapatite Precursors

The terms “biomimetic hydroxyapatite precursors” refer to the precursor ions leading to the formation of biomimetic hydroxyapatite for instance under conditions described in Nassif et al., Chemistry of Materials, 22 (12), pp.3653-3663, 2010.

Suitable biomimetic hydroxyapatite precursors include CaCl₂·2H₂O, NaH₂PO₄ and NaHCO₃ and salts that may be found in the mineral bone composition including salts of magnesium, zinc, fluor and strontium.

The molar ratio Ca/P typically ranges from 1.5 to 2.

The calcium to phosphate plus carbonate ratio (Ca/[P+C]) molar ratio is consistent with the formation of hydroxyapatite preferably with a formula of Ca₁₀₋ₓ(PO₄)₆₋ₓ(CO₃)ₓ(OH)₂₋ₓ with 0≤x≤2 (von euw, scientific report 2019).

### Amorphous Calcium Phosphate

The terms “amorphous calcium phosphate” refer to amorphous calcium phosphate particles. The amorphous calcium phosphate is typically in the form of powder.

The amorphous calcium phosphate powder may be synthesized by the atomization of the biomimetic hydroxyapatite precursors acidic solution using a spray-processing technology as disclosed in WO2016/146954.

### Aqueous Solvent

The aqueous solvent may be any physiologically compatible aqueous solvents. Non limitative examples of suitable aqueous solvents include physiological serum, phosphate buffer, sodium bicarbonate, sterile water, normal saline, blood or blood plasma.

### Optional Therapeutic or Bioactive Agents

The compositions may comprise one or more therapeutic or bioactive agents, such as for example anti-inflammatory agents, antibiotics, osteogenic proteins, hyaluronic acid, cells, growth factors, and anti-osteoporotic agents (e.g. salts).

### Processes for Preparing the Compositions

### Process 1: Mixing Dense Collagen Microparticles and Hydroxyapatite or Amorphous Calcium Phosphate

The compositions according to the invention may be prepared by mixing a desired weight of dense collagen microparticles, typically in the form of powder, with a desired weight of hydroxyapatite powder or amorphous calcium phosphate powder (process 1). The dense collagen microparticles and the hydroxyapatite or amorphous calcium phosphate powder may be prepared as described herein above. The dense collagen microparticles and the hydroxyapatite or amorphous calcium phosphate powder are typically mixed in a mortar. The mixing of the dense collagen microparticles and hydroxyapatite or amorphous calcium phosphate powder is typically made in a weight ratio that is suitably chosen to reproduce the targeted tissue and which can be adapted to the targeted application. Non-limiting examples of suitable dense collagen microparticles to hydroxyapatite or amorphous calcium phosphate powder weight ratio include the following ratios: from 10:90 to 90:10, preferably from 30:70 to 80:20, more preferably 50:50.

After the dense collagen microparticles and the hydroxyapatite or amorphous calcium phosphate powder have been mixed in a suitable weight ratio, an aqueous solvent as described herein above is added to the mixture. The weight ratio of the aqueous solvent to the mixture of dense collagen microparticles and hydroxyapatite or amorphous calcium phosphate powder typically ranges from 1.8 to 10 (i.e. in the range from 0.18 mL to 1 mL of solvent per 100 mg of the mixture of dense collagen microparticles and hydroxyapatite or amorphous calcium phosphate powder), preferably from 2 to 9, more preferably from 3 to 8. The mixture may then be supplemented with one or more therapeutic or bioactive agents, such as anti-inflammatory or anti-osteoporotic agents.

After mixing, the obtained composition, in a paste or liquid form, may be inserted in a sterile syringe.

All steps of the disclosed process are preferably performed in sterile conditions.

The syringe may then be stored in a dry place at a temperature lower than the denaturation temperature of the collagen, preferably in a fridge at 4° C.

Alternatively, the compositions may be prepared by atomizing an acidic solution comprising biomimetic hydroxyapatite precursors and non-denatured and uncrosslinked collagen (process 2) or the dense collagen microparticles may be mixed with an aqueous solution containing the biomimetic hydroxyapatite precursors (process 3).

### Process 2: Atomization of Biomimetic Hydroxyapatite Precursors Containing Collagen Solution

The compositions according to the invention may be prepared by a process comprising the step of atomizing of a solution containing hydroxyapatite precursors and non-denatured and uncrosslinked collagen.

The spray-processing technology is performed as disclosed in WO2016/146954. The atomization is performed with an acid-soluble collagen solution (non-denatured and uncrosslinked collagen). The concentration of collagen in the acidic collagen solution typically ranges from 0.1 to 10 mg/L. The acidic collagen solution has a pH inferior to 7. The acid is typically acetic acid. The acetic acid concentration in the acidic collagen solution typically ranges from 0.1 to 1000 mM. The collagen solution is mixed with a desired volume/concentration of a biomimetic hydroxyapatite precursors solution (i.e. the acidic collagen solution is supplemented with the ionic precursors of hydroxyapatite). In a preferred set-up, the biomimetic hydroxyapatite precursors solution is made by dissolving biomimetic hydroxyapatite platelets in an acidic solution.

Atomization is typically performed at a temperature below about 40° C., in particular below about 39° C., 38° C. or 37° C., to obtain a non-denatured powdered composition.

The microparticles resulting from the atomization are referred herein as “hybrid dense collagen microparticles”. The hybrid dense collagen microparticles are dense collagen microparticles as disclosed herein above containing biomimetic hydroxyapatite ionic precursors (e.g. CaCl₂·2H₂O, NaH₂PO₄ and NaHCO₃). Hybrid microparticles with different ionic compositions may be obtained. Calcium acetate can be used as an alternative to calcium chloride to avoid NaCl precipitation.

The mixing of the hybrid dense collagen microparticles and the physiologically compatible aqueous solvent (containing or not biomimetic hydroxyapatite precursors) is typically made in a weight ratio that is suitably chosen to reproduce the targeted tissue and which can be adapted to the targeted application.

After mixing, the obtained composition, in a paste or liquid form, may be inserted in a sterile syringe.

All steps of the disclosed process are preferably performed in sterile conditions. The syringe may then be stored in a dry place at a temperature lower than the denaturation temperature of the collagen, preferably in a fridge at 4° C.

Process 3: Mixing Dense Collagen Microparticles with Biomimetic Hydroxyapatite Precursors Solution

The compositions according to the invention may be prepared by mixing a desired weight of dense collagen microparticles, typically in the form of powder, with a desired volume of a biomimetic hydroxyapatite precursors solution. The dense collagen microparticles and the biomimetic hydroxyapatite precursors solution may be prepared as described herein above. The dense collagen microparticles and the biomimetic hydroxyapatite precursors solution are typically mixed in a mortar or well. The mixing of the dense collagen microparticles and the biomimetic hydroxyapatite precursors solution is typically made in a weight ratio that is suitably chosen to reproduce the targeted tissue and which can be adapted to the targeted application.

The volume of biomimetic hydroxyapatite precursors solution added to the dense collagen microparticles typically leads to a final concentration of 80 mg/ml of collagen. After mixing, the obtained composition, in a paste or liquid form, may be inserted in a sterile syringe. All steps of the disclosed process are preferably performed in sterile conditions.

The syringe may then be stored in a dry place at a temperature lower than the denaturation temperature of the collagen, preferably in a fridge at 4° C.

### Compositions and Uses Thereof

The compositions of the present invention comprise:
dense collagen microparticles (i.e. microparticles comprising more than 90 wt % of collagen);
biomimetic hydroxyapatite or biomimetic hydroxyapatite precursors or amorphous calcium phosphate; and
a physiologically compatible aqueous solvent.

When the compositions are prepared in accordance with process 1, the compositions of the present invention may be more specifically defined as comprising:
dense collagen microparticles (i.e. microparticles comprising more than 90 wt % of collagen);
biomimetic hydroxyapatite platelets or amorphous calcium phosphate; and
a physiologically compatible aqueous solvent.

When the compositions are prepared in accordance with process 2, the compositions of the present invention may be more specifically defined as comprising:
hybrid dense collagen microparticles (i.e. dense collagen microparticles comprising biomimetic hydroxyapatite precursors); and
a physiologically compatible aqueous solvent that optionally comprise biomimetic hydroxyapatite precursors.

When the compositions are prepared in accordance with process 3, the compositions of the present invention may be more specifically defined as comprising:
dense collagen microparticles (i.e. microparticles comprising more than 90 wt % of collagen); and
a physiologically compatible aqueous solvent comprising biomimetic hydroxyapatite precursors;

The compositions typically comprise from 10 mg to 100 mg of dense collagen microparticles per mL of composition, preferably from 40 mg to 80 mg, more preferably from 50 mg to 70 mg.

In some embodiments, the weight ratio of dense collagen microparticles to biomimetic hydroxyapatite or amorphous calcium phosphate ranges from 10:90 to 90:10, preferably 30:70 to 80:20, more preferably is 50:50, in the compositions (that may be prepared in accordance with process 1).

The skilled person will readily adjust the weight ratio of dense collagen microparticles to biomimetic hydroxyapatite or amorphous calcium phosphate to adapt the formulation of the compositions to the envisioned uses, targeted mineralized tissue and administration sites. By varying the weight ratio of dense collagen microparticle to biomimetic hydroxyapatite, mineralized collagen matrices with different ultrastructures and mechanical properties may be obtained.

The compositions of the present invention can be readily implanted or injected or otherwise applied to a site in which there is a need for a mineralized tissue repair. For instance, the compositions can be suitably injected with a syringe directly at the site of the defect to be repaired. The compositions have the ability to fill the targeted defect and take the same 3D shape. The compositions are sufficiently adhesive/tacky to hold in place in the defect without external assistance or agents.

Alternatively, the compositions can be injected in a mold to form a preformed matrix. The preformed matrix is implantable.

Thanks to their properties and abilities to mimic different types of mineralized biological tissues, the compositions of the present invention may be used for mineralized tissues repair and regeneration. In particular, the compositions may be used for bone repair and regeneration. They may be used for inducing new bone formation, promoting bone growth and/or treating bone defects. A variety of bone defects in which new bone formation or growth is required may be treated with the disclosed compositions. More specifically, the compositions of the present invention may be used as bone and/or dental substitutes. For instance, the compositions may be used for tooth filling in dental surgery. They may also be used for repairing bones defects in bone reconstructive procedure, for instance in maxillofacial surgery (reconstruction of the bony arch of teeth) or in orthopedic surgery, in particular for vertebroplasty. In these embodiments, the compositions are directly injected at the site of the defects to be repaired.

In some embodiments, the invention relates to a method for regenerating mineralized tissue, inducing new bone formation, promoting bone growth and/or treating bone defects which comprises the steps of injecting or implanting a composition as described herein in an individual in need of treatment thereof.

The compositions of the present invention may also be used to prepare preformed mineralized collagen matrices, such as preformed bone-like materials. For instance, the compositions may be injected in a mold or be used for 3D-printing to form a preformed implantable mineralized collagen matrix. The present invention also relates to these preformed implantable matrices.

The compositions may be used to induce the biomimetic remineralization of osteoporotic bone and locally deliver Sr²⁺, which can restore the unbalance between bone formation by osteoblasts and bone resorption by osteoclasts; one of the main responsible for osteoporosis. For example, the hybrid collagen/CHA ionic precursor microparticles can be injected and induce in situ the mineralization upon contact with the body fluid. Moreover, the incorporation of Sr²⁺ in this composition may be used for the in situ release of this ion, which is usually present in oral formulation for osteoporosis treatments i.e. strontium ranelate.

Embodiments of the present invention will now be described by way of the following examples which are provided for illustrative purposes only, and not intended to limit the scope of the disclosure.

### EXAMPLES

### Example 1

### Injectable and Pre-Formed Hybrid Material (Collagen/Hydroxyapatite Ratio 50:50) in 0.9% Saline

### Synthesis of Carbonated Hydroxyapatite

The synthesis of carbonated hydroxyapatite was performed in accordance with the procedure described by Nassif et al. (Chemistry of Materials, 22 (12), pp.3653-3663, 2010). A solution of 110 mM CaCl₂·2H₂O, 33 NaH₂PO₄ and NaHCO₃ was prepared in 500 mM acetic acid. The pH was adjusted to 2.2 with HCl solution at 37%. Two flasks (35 mL) were filled with 20 ml of this solution and placed in a hermetically sealed chamber (i.e. put in a 1 L beaker covered with paraffin), in the presence of a third vial containing 8 mL of an aqueous solution of NH3 28-30% by mass. Before closing, these 3 flasks were covered with parafilm pierced with 6 holes using a needle in order to slow down the gaseous diffusion of the ammonia. The device was then left for 6 days. Then, the precipitate was collected by centrifugation at room temperature (20 minutes at 6000 rpm), washed with ultrapure water until the pH of the supernatant is close to that of the washing water. The white powder obtained was finally dried in an oven at 37° C. for 7 days. The dry powder was then finely milled in a mortar with a pestle to obtain a fine powder.

### Synthesis of Collagen Microparticles by Aerosol

The synthesis of collagen microparticles was performed in accordance with the procedure described by Nassif et al. (Paris, 2018. Injectable collagen suspensions, the preparation method thereof, and the uses thereof, particularly for forming dense collagen matrices. U.S. patent application Ser. No. 15/558,787) and Lama et al. (Self-Assembled Collagen Microparticles by Aerosol as a Versatile Platform for Injectable Anisotropic Materials. Small, p. 1902224, 2019).

A collagen solution concentrated to 1.2 mg/mL was obtained by diluting a collagen stock solution (usually 1.3 to 5 mg/mL) in acetic acid (500 mM). 250 mL of said solution was dried in a spray-dryer (Büchi B290). The spray-dryer was placed under a fume hood next to a mobile reversible air conditioner. The temperature under the fume hood should ideally be maintained between 19° C. and 21° C. (unfavorably above 25° C.). The injection speed of the collagen solution (at 1.2 mg/mL) was controlled by the peristaltic pump of the atomizer and was equal to 0.6 mL/min. The set temperature of the nozzle is maintained at 30° C. The actual temperature of the nozzle oscillates between 34° C. and 35° C. after one hour of stabilization at vacuum (before starting the peristaltic pump). The internal temperature of the system, measured between the drying column and the particle collection cyclone, is between 19° C. and 25° C. The air flow responsible for droplet shearing at the nozzle outlet is 414 L/h. The suction power, which controls the drying of the droplets between the nozzle outlet and the collector, is set at 50% of the maximum capacity of the drying system, i.e. 20 m3/h. The “nozzle” parameter, which is used to prevent coagulation of the solution at the end of the nozzle, is set at 2. Aluminum is placed on both sides of the joint between the column and the cyclone to avoid heat loss as much as possible. The formed particles are collected by a high-performance cyclone connected to a flask. In order to recover all the powder remaining on the walls of the cyclone and to maximize the yield, the temperature set point is turned off at the end of the atomization and the suction is increased in 10% steps, from 50% (20 m3/h) to 100% (40 m3/h) by waiting 5 minutes per step. The process efficiency is between 50% and 60%. To ensure sterile conditions, a commercial device of filters of different sizes sold by BEKO technologies can be used. It is also recommended to sterilize the whole setup with >94° ethanol before spraying the collagen.

### Preparation of the Injectable Composition

60 mg of the collagen powder obtained as disclosed herein above and 60 mg of hydroxyapatite powder obtained as disclosed herein were mixed in a mortar. 1 mL of sterile saline (NaCl 0.9%) was added in the mortar. The whole was mixed for about one minute to obtain a homogeneous paste. The paste was transferred into an empty 1 mL syringe. The plunger was put back in place. The paste was then ready to be injected into the defect.

### Preparation of the Pre-Formed Matrices

The above protocol is repeated. The mixture is injected through the syringe into a silicone mold of the desired dimensions and total volume of 1 mL. Fibrillogenesis (gelation) is performed under ammonia vapor overnight. The gel is then removed from the mold and rinsed with saline to until reaching neutral pH. The material can then be implanted in a cavity corresponding to the shape of the mold.

### Characterization of the Injectable and Pre-Formed Materials

### Methods:

Thermogravimetric analysis (TGA): Experiments were performed with a NIETZSCH STA 409PC instrument on a thermo-microbalance under an oxidizing atmosphere from room temperature to 850° C. with a heating rate of 5° C./min.

Differential scanning calorimetry (DSC): Experiments were performed with a TA Q-20 machine. The heating rate was set at 5° C./min and the temperature range from 20° C. to 80° C. About 20 mg piece of material was weighed and placed in a sealed aluminum pan. An empty sealed aluminum pan was used a reference.

Polarized light microscopy (PLM): The materials were placed without any treatment between a glass slide and a coverslip. Observations were made using a transmission Zeiss Axiolmager A2 POL. The microscope is equipped with the standard accessories for examination of birefringent samples under polarized light (i.e. crossed polarizers) and an AxioCam CCD camera.

Scanning electron microscopy (SEM): Samples were fixed in 2.5% glutaraldehyde solution. After washing in cacodylate/saccharose buffer solution, they were dehydrated through ethanol baths (from 30% to 100% ethanol). Supercritical CO2 drying was performed by a CPD-300 (Leica). Dried samples were cut into pieces, put on carbon tape covering sample holders, covered with 15 nm gold layer. Observations were carried out by using a Hitachi S-3400 N microscope operating at 3 kV and 30 pA.

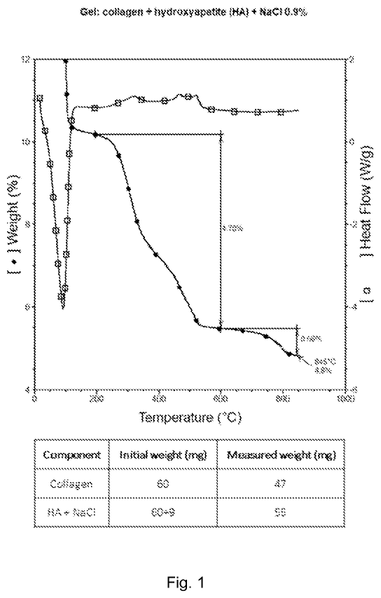

The final composition of the materials is consistent with that of initial mixture, taking into account the presence of water in the collagen microparticles (about 10%) (FIG. 1).

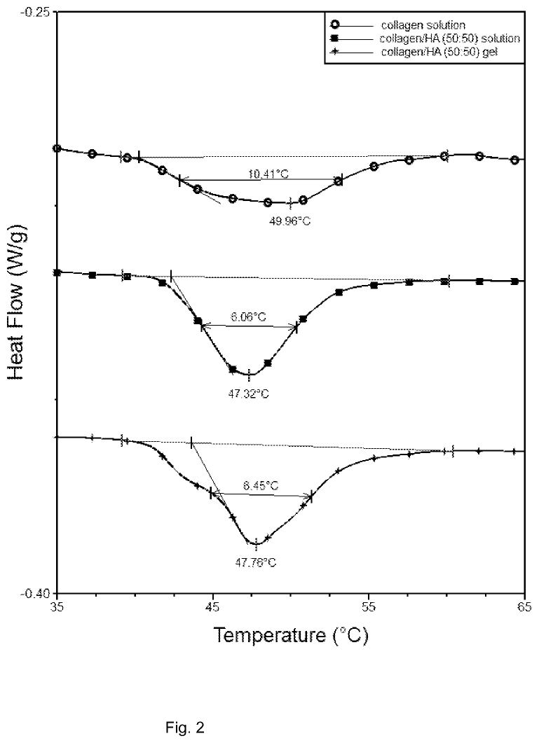

The denaturation temperature of collagen is about 48° C. This is close to the denaturation temperature reported for collagen gels (Tiktopulo, E. I. and Kajava, A. V. (1998) ‘Denaturation of type I collagen fibrils is an endothermic process accompanied by a noticeable change in the partial heat capacity’, Biochemistry, 37 (22), pp. 8147-8152) indicating that the addition of saline can promote fibrillogenesis. Indeed, the denaturation temperature remains unchanged when fibrillogenesis is induced by ammonia vapors (mineralized collagen gel). The addition of hydroxyapatite to the collagen microparticles and saline mixture seems to induce favorable interactions: the denaturation enthalpy is higher and the width at mid-height of the endotherm is less important (FIG. 2). This means that the addition of hydroxyapatite would tend to homogenize the collagen fibril (or microfibril) population.

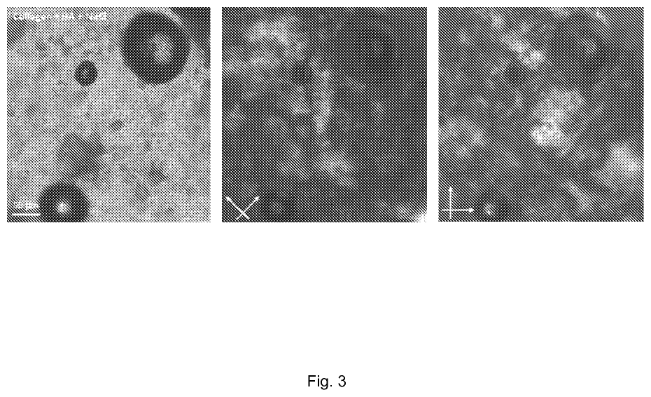

As observed by PLM (FIG. 3), the solution exhibits domains of birefringence testifying the anisotropy of the material, and confirming that the addition of hydroxyapatite under these conditions does not prevent the self-assembly of collagen in liquid crystal phases.

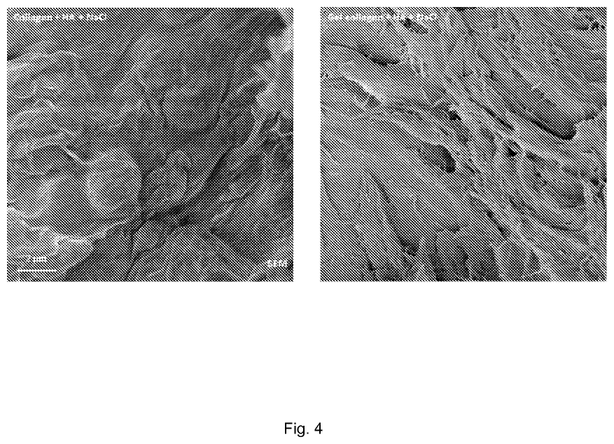

This local anisotropy can also be seen by SEM through the observation of aligned mineralized collagen fibril groups (FIG. 4). Before fibrillogenesis, the material also shows partially dissolved collagen microparticles. The dissolution of the microparticles can be modulated by the mixing time before injection. After fibrillogenesis, more defined fibrils are observed.

### Example 2

### Pre-Formed Hyybrid Material (Collagen/Hydroxyapatite Ratio 50:50) in 2 mM Acetic Acid

### Preparation of the Pre-Formed Hybrid Material

40 mg of the collagen powder obtained as disclosed herein above and 40 mg of the hydroxyapatite powder obtained as disclosed herein above are mixed in a mortar. 0.15 mL of 2 mM acetic acid is added to the mortar. The whole is mixed for about one minute to obtain a homogeneous paste. The paste can be injected via a 1 ml syringe into a mold or spread into a mold with a spatula. Fibrillogenesis is performed under ammonia vapors for three hours. The gel is then demolded and rinsed with PBS until reaching neutral pH. The material can then be implanted in a cavity corresponding to the shape of the mold.

### Characterization of the Pre-Formed Hybrid Material

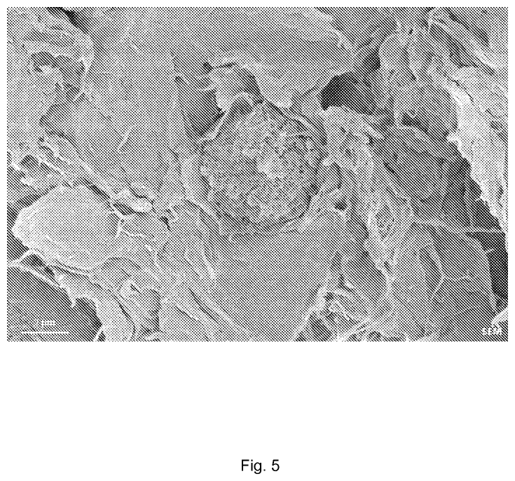

SEM observation shows fibrillar alignment domains (FIG. 5). The material appears dense.

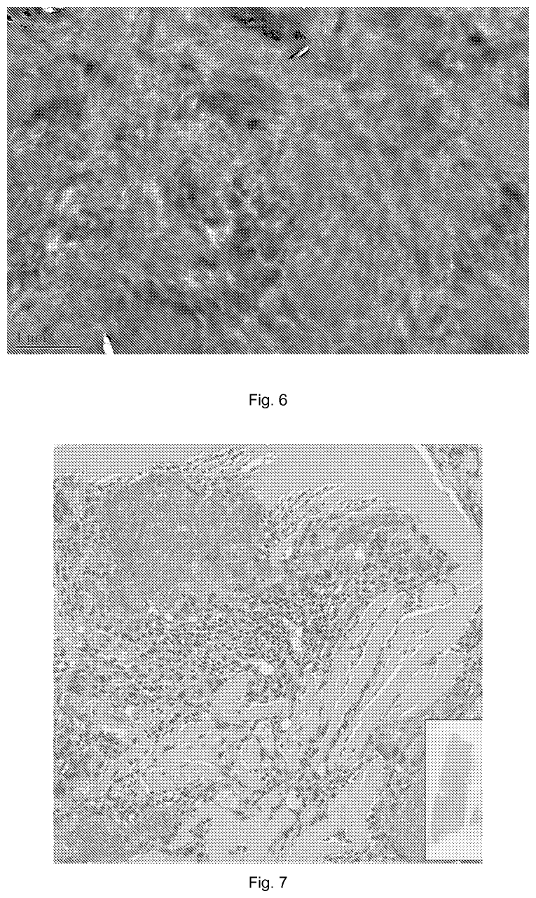

SEM reveals areas of co-alignment of collagen fibrils and hydroxyapatite nanoplatelets, resembling those observed in compact bone (FIG. 6).

### Example 3

### Injection in a Cavity—In Vivo Data

The biocompatibility of injectable collagen matrices was tested in intramuscular position in rat. For this purpose, a skin incision was made along the femoral axis and the fascia over the biceps femoris and gluteal muscle was incised. A gap was created between the 2 muscles to insert the material of example 2 by injection. At 30 days post-surgery, the rats were euthanized, the implanted materials were extracted and analyzed. Histological thin section stained by hematoxylin-eosin (FIG. 7) show both the infiltration of immune cells and the colonization by cells of the mesenchymal lineage (fibroblasts or stem cells) confirming the malleability, simplicity and non-toxicity of the injectable materials.

### Example 4

### Injectable and Pre-Formable Material (Dense Collagen Microparticles Mixed with Biomimetic Hydroxyapatite Precursors Solution)

### Preparation of the Injectable Composition

90 mg of the collagen powder obtained as disclosed herein above was mixed with 1 ml of biomimetic hydroxyapatite precursors solution obtained as disclosed herein (110 mM CaCl₂·2H₂O, 33 mM NaH₂PO₄ and 33 mM NaHCO₃) was prepared in 500 mM acetic acid The whole was mixed for about one minute to obtain a homogeneous paste. The paste was transferred into an empty 1 ml syringe. The plunger was put back in place. The paste was then ready to be injected into the defect.

### Preparation of the Pre-Formed Matrix

The above protocol is repeated. The mixture is injected through the syringe into a silicone mold of the desired dimensions and total volume of 1 mL. Fibrillogenesis (gelation) is performed under ammonia vapor overnight. The gel is then removed from the mold and rinsed with saline to until reaching neutral pH. The material can then be implanted in a cavity corresponding to the shape of the mold.

### Characterization of the Pre-Formed Material

Methods:

Scanning electron microscopy (SEM): Samples were fixed in 2.5% glutaraldehyde solution. After washing in cacodylate/saccharose buffer solution, they were dehydrated through ethanol baths (from 30% to 100% ethanol). Supercritical CO2 drying was performed by a CPD-300 (Leica). Dried samples were cut into pieces, put on carbon tape covering sample holders, covered with 15 nm gold layer. Observations were carried out by using a Hitachi S-3400 N microscope operating at 3 kV and 30 pA.

Transmission electron microscopy (TEM): This protocol is similar to the protocol for the SEM. Then, sample was rinsed, dehydrated, and embedded in Epon 812. Sections (˜80 nm) were observed with a Tecnai spirit G2 operating at 120 kV

Fourier-transform infrared spectroscopy: Fourier-transform infrared spectra with attenuated total reflectance were obtained on a Perkin Elmer Spectrum One spectrophotometer with a resolution of 1 cm⁻¹.

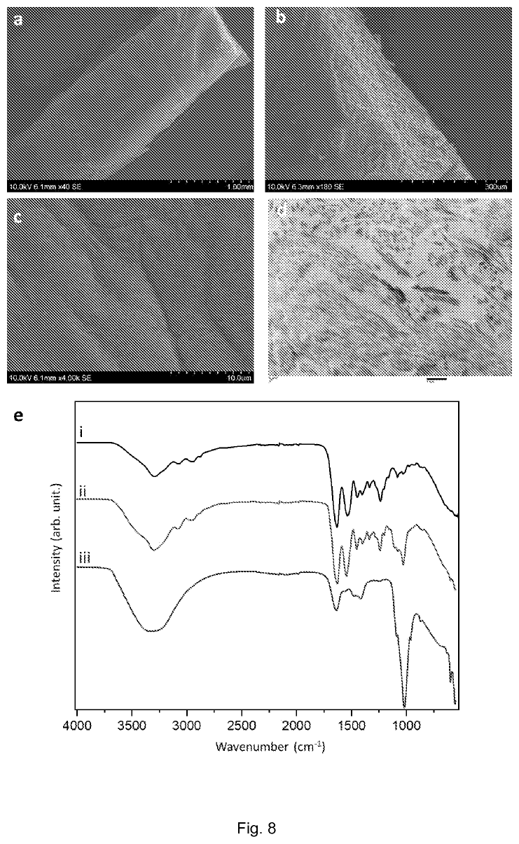

SEM micrographs show that the material displays a high density of collagen fibrils (FIG. 8 a-c).

TEM micrograph shows the alignment of collagen fibrils in the material (FIG. 8d).

Infrared spectrum of the pre-formed material displays vibrational bands ascribed to both collagen and apatite, thus confirming the formation of a collagen mineralized matrix (FIG. 8e)

### Example 5

### Synthesis of Hybrid Collagen Microparticles by Aerosol

The synthesis of collagen microparticles was performed in accordance with the procedure described by Nassif et al. (Paris, 2018. Injectable collagen suspensions, the preparation method thereof, and the uses thereof, particularly for forming dense collagen matrices. U.S. patent application Ser. No. 15/558,787) and Lama et al. (Self-Assembled Collagen Microparticles by Aerosol as a Versatile Platform for Injectable Anisotropic Materials. Small, p. 1902224, 2019). In addition, the salts present in biomimetic hydroxyapatite precursor were added to the low concentration collagen acidic collagen solution before the atomization leading to the final composition: 2 mg/mL collagen, 500 mM acetic acid, 110 mM CaCl₂·2H₂O, 33 NaH₂PO₄ and NaHCO₃ The composition of ionic precursors can be modified to form hybrid collagen microparticles with different mineral/collagen ratios, and loaded with different therapeutic ions e.g. Sr²⁺, Mg²⁺, Zn²⁺.

For example, SrCl₂·6H₂O may be added to the biomimetic hydroxyapatite precursors solution to obtain a 10% Sr²⁺ in relation to Ca²⁺ (mol/mol).

### Preparation of the Injectable Composition

90 mg of the collagen powder obtained as disclosed herein above was mixed with 1 ml of 500 mM acetic acid. The whole was mixed for about one minute to obtain a homogeneous paste. The paste was transferred into an empty 1 mL syringe. The plunger was put back in place. The paste was then ready to be injected into the defect. Different weight of ionic may be used to obtain different mineral/collagen ratios.

### Preparation of the Pre-Formed Matrix

The above protocol is repeated. The mixture is injected through the syringe into a silicone mold of the desired dimensions and total volume of 1 mL. Fibrillogenesis (gelation) is performed under ammonia vapor overnight. The gel is then removed from the mold and rinsed with saline to until reaching neutral pH. The material can then be implanted in a cavity corresponding to the shape of the mold.

### Characterization of the Pre-Formed Material

### Methods:

Energy-dispersive X-ray spectroscopy (EDX) microanalysis: The EDX instrument X-Max (Oxford Instruments) was coupled to a scanning electron microscope Hitachi S-3400 N operating at 12 kV, and the Oxford Microanalysis Group XAN.70 software was used for this analysis. Dried samples were cut into pieces, put on carbon tape covering sample holders, covered with 15 nm carbon layer.

Hybrid dense collagen/biomimetic hydroxyapatite precursors microparticles (process 3).

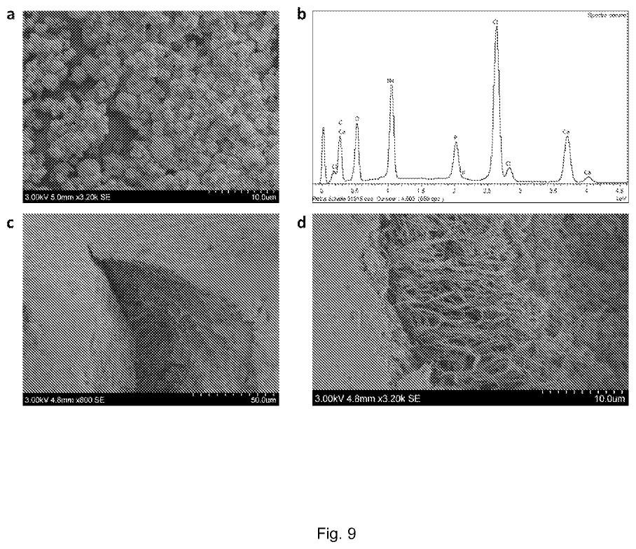

As observed in the SEM micrograph dense microparticles were formed (FIG. 9a).

EDX spectrum confirms the incorporation of biomimetic hydroxyapatite precursors in the dense collagen microparticles (FIG. 9b).

SEM images show a high density of fibrils in the pre-formed material produced using the hybrid dense collagen/biomimetic hydroxyapatite precursors microparticles (FIG. 9c-d).

### Example 6

### Injectable hybrid material (collagen/amorphous calcium phosphate ratio 30:70) in acetic acid 2 mM

Preparation of Amorphous calcium phosphate

The amorphous calcium phosphate powder is synthesized by atomization of a biomimetic hydroxyapatite precursors acidic solution of 110 mM CaCl₂·2H₂O, 33 mM NaH₂PO₄ and 33 mM NaHCO₃ in 500 mM acetic acid using a spray-processing technology as disclosed in WO2016/146954.

### Preparation of the Pre-Formed Hybrid Material

40 mg of the collagen powder obtained as disclosed herein above and 40 mg of the amorphous calcium phosphate powder obtained as disclosed herein above are mixed in a mortar. 0.15 mL of 2 mM acetic acid is added to the mortar. The whole is mixed for about one minute to obtain a homogeneous paste. The paste can be injected via a 1 mL syringe into a mold or spread into a mold with a spatula. Fibrillogenesis is performed under ammonia vapors for three hours. The gel is then demolded and rinsed with PBS until reaching neutral pH. The material can then be implanted in a cavity corresponding to the shape of the mold.

1. A composition comprising:
collagen microparticles comprising more than 90% by weight of collagen;
biomimetic hydroxyapatite or biomimetic hydroxyapatite precursors or amorphous calcium phosphate; and
a physiologically compatible aqueous solvent.

2. The composition according to claim 1 wherein the collagen microparticles have a diameter ranging from 0.05 to 20 μm as measured by electron microscopy or by dynamic light scattering.

3. The composition according to claim 1 wherein the collagen microparticles are type I collagen microparticles.

4. The composition of claim 1 wherein the physiologically compatible aqueous solvent is physiological serum, phosphate buffer, sodium bicarbonate or blood.

5. The composition according to claim 1 wherein the composition further comprises one or more therapeutic or bioactive agents.

6. The composition of claim 1 wherein the collagen microparticles to biomimetic hydroxyapatite or amorphous calcium phosphate weight ratio ranges from 10:90 to 90:10.

7. The composition of claim 1 wherein the composition is injectable or implantable.

8. A method for repairing and regenerating mineralized tissue, comprising injecting or implanting an effective amount of the composition of claim 1 in an individual in need of treatment thereof.

9. The method of claim 8, wherein the mineralized tissue is a bone.

10. A method for inducing new bone formation, promoting bone growth and/or treating bone defects, comprising injecting or implanting an effective amount of the composition of claim 1 in an individual in need of treatment thereof.

11. A method for repairing bones defects in bone reconstructive procedure comprising injecting or implanting an effective amount of the composition of claim 1 in an individual in need of treatment thereof.

12. A preformed implantable matrix comprising the composition of claim 1.

13. A process for preparing the composition of claim 1, the process comprising the steps of:
(a) providing collagen microparticles comprising more than 90% by weight of collagen;
(b) providing biomimetic hydroxyapatite powder or amorphous calcium phosphate powder;
(c) mixing the collagen microparticles and the hydroxyapatite powder or amorphous calcium phosphate powder; and
(d) adding a physiologically compatible aqueous solvent.

14. A process for preparing the composition of claim 1, the process comprising the steps of:
(a) atomizing an acidic solution comprising non-denatured and uncrosslinked collagen and biomimetic hydroxyapatite precursors;
(b) adding a physiologically compatible aqueous solvent to the particles obtained in step (a).

15. A process for preparing the composition of claim 1, the process comprising the steps of:
(a) providing collagen microparticles comprising more than 90% by weight of collagen;
(b) adding a physiologically compatible aqueous solvent comprising biomimetic hydroxyapatite precursors to the collagen microparticles.

16. The composition of claim 6, wherein the collagen microparticles to biomimetic hydroxyapatite or amorphous calcium phosphate weight ratio ranges from 30:70 to 80:20.

17. The composition of claim 6, wherein the collagen microparticles to biomimetic hydroxyapatite or amorphous calcium phosphate weight ratio is 50:50.

18. The method of claim 11, wherein the bone reconstructive procedure is maxillofacial surgery or orthopaedic surgery.
